(19) United States
(12) Patent Application Publication
(10) Pub. No.: US 2009/0253754 A1
(43) Pub. Date: Oct. 8, 2009

(54) Fast dissolving films and coatings for controlled release of flavors, active pharmaceutical ingredients, food substances, and nicotine

(76) Inventors: Francesca Selmin, Milan (IT); Paolo Blasi, Belmonte Piceno (IT); David R. Worthen, Lexington, KY (US); David Johnson, Owensboro, KY (US); Patrick P. DeLuca, Lexington, KY (US)

Correspondence Address: Krieg DeVault LLP, Suite 2800, One Indiana Square, Indianapolis, IN, 46204, US

(21) Appl. No.: 12/315,603

(22) Filed: Dec. 4, 2008

Related U.S. Application Data
(60) Provisional application No. 61/005,289, filed on Dec. 4, 2007.

(51) Int. Cl. A61K 31/44; A24B 15/36; B05D 3/02; A61K 47/30; A61P 43/00; C08L 1/00; C08L 1/26; A23L 1/226

(52) U.S. Cl. 514/343; 131/277; 427/384; 514/777; 106/163.01; 106/172.1; 426/650

(57) ABSTRACT

A fast-dissolving film for use as a platform for the delivery of material to the oral cavity, comprising a film forming agent; a plasticizing agent; and a fast-dissolving, water-soluble agent; and methods for producing same.

This application claims the benefit of the filing date of U.S. Provisional Patent Application Ser. No. 61/005,289, filed Dec. 4, 2007, pursuant to 35 U.S.C. § 120.

### FIELD OF THE INVENTION

The present invention relates to edible films generally, and more particularly to edible films that are fast-dissolving when they come into contact with water-based aqueous systems such as human saliva, and that provide controlled release of flavors, active pharmaceutical ingredients, foods substances, or nicotine derivatives.

### BACKGROUND OF THE INVENTION

Films for pharmaceutical or food use have been known for some time. For example, compositions based on edible films are already commercially available, such as, for example, LISTERINE® brand breath freshening dissolving strips. Most of these existing products are based on pullulan as the filmogenic component. See, e.g., U.S. Pat. Nos. 4,623,394; 5,411,945; 5,518,902. Pullulan is an expensive component, and it is not readily available. Other materials have been used in place of pullulan, including modified starches, such as maltodextrin, and starch derivatives, as described, for example, in WO 2005039543 and EP 1417895A1, respectively. However, these prior art films are based on very hygroscopic film-forming materials and on expensive packaging, both in terms of materials and technologies. Further, the use of additional ingredients is compulsory to preserve their features over different storage conditions.

Fast-dissolving drug delivery systems, such as fast-dissolving tablets and films, are gaining interest because they overcome the disadvantages associated with liquid and conventional dosage forms, which is improving patients' compliance and dosage accuracy. See, e.g., U.S. Pat. No. 4,925,670. Fast-dissolving films comprised of water-soluble polysaccharides, appropriate excipients, and flavors have been successfully employed in a variety of consumer breath freshening products, such as LISTERINE POCKETPACKS®. A number of researchers have developed buccal drug delivery systems using similar formulations. These materials are also employed in various types of food packaging and food wrapping. Depending upon their composition, such films may possess superior flavor retention characteristics with low water vapor penetration. See, e.g., PCT Int. Appl. (2005) WO 2005046363 (U.S. Smokeless Tobacco Company); U.S. Pat. No. 5,411,945; U.S. Pat. No. 4,018,233. These characteristics might be particularly useful if these films were ever employed to package snuff in individual packets for buccal use. Indeed, certain tobacco-polysaccharide composites have already been developed using fast-dissolving polysaccharides. See, e.g., U.S. Pat. No. 5,411,945; U.S. Pat. No. 4,018,233.

U.S. Patent Application No. 2007/0154542 relates to pharmaceutical compositions comprising one or more non-steroidal anti-inflammatory drugs and one or more acid inhibitors as the active agents. In one embodiment, the invention claimed relates to orally dissolvable films in which the proton pump inhibitor is enteric coated on dispersed fine particulates. The invention claims, without limitation, the use of surfactants and plasticizers, polyalcohols; and thermo-setting gels such as pectin, carageenan, and gelatin, all of which can help to maintain the dispersion of components. Citric acid, or another suitable agent, can be added to stimulate saliva production and facilitate rapid dissolution of the film in the oral cavity, see, e.g., U.S. Pat. No. 4,820,506, and/or provide an acidic environment for an enteric coated proton pump inhibitor. Additional ingredients can be incorporated into the films of the claimed invention include, without limitation, colorants, flavors, fragrances, mouthwash components, preservatives, sweetening agents, vitamins and combinations thereof.

U.S. Pat. No. 4,197,289 claims a solid pharmaceutical unit dosage form comprising a plurality of layers of an edible, therapeutically inert web consisting of a polymeric composition organic film forming ingredient, a plasticizer, and one or more medicaments in fine particles. The dosage forms have a consistency of release of medicament that can be controlled to exacting specifications. The disclosed solid dosage forms are prepared by high speed automated equipment. The need of the high speed automated equipment claimed in the invention is a clear disadvantage of this patent.

A novel molded article exhibiting a gradual disintegration effect, prepared using pullulan, is claimed in U.S. Pat. No. 4,623,394. The term “molded article(s)” is used for various two- or three-dimensional molded articles (e.g., granule, fiber, filament, rod, gauze, cloth, film, sheet, paper, coating membrane, tube, capsule, tablet, sponge, laminated article). The molded article is advantageously usable for industrial materials, pharmaceuticals, consumers' products. The use of pullulan to produce food, pharmaceutical, cosmetic and agricultural products is also claimed in U.S. Pat. No. 5,518,902.

A therapeutic dosage form made of anhydrous but hydratable monolithic polymer matrix (i.e., polyethylene glycol), amorphous fumed silica, as well as a therapeutic agent, is claimed in U.S. Pat. No. 5,047,244. The dosage form contains a mucoadhesive face, and a water-insoluble barrier layer, the non-adhesive face.

U.S. Pat. No. 5,284,659 describes a confectionary compressed tablet designed to dissolve in the oral cavity that contain a flavor ingredient intimately bound with a bioadhesive material. The invention discloses the use of different flavors, including oil of wintergreen (methyl salicylate), and a group of bioadhesive materials, such as amylopectin, carboxymethylcelluloses, hydroxyethylcelluloses, acrylates, gelatin, guar gum, karaya gum, tragacanth, agar, alginic acid, dextran, methylcellulose, pectin, polyethylene glycol, polyvinylpyrrolidone and mixtures thereof.

Rapidly disintegrating sheet-like presentations of multiple dosage units are described in U.S. Pat. No. 5,629,003. The presentation is characterized by the fact that it comprises a mass of at least 20 to 60%-wt. of a film former, at least 2 to 40%-wt. of a gel former, at least 0.1 to 35%-wt. of an active substance, and up to 40%-wt. of an inert filling agent. The invention also discloses the use of up to 30%-wt. of a polar solvent, and includes processing to form a homogeneous, spreadable or extrudable mass.

U.S. Pat. Nos. 5,948,430; 6,177,096 and 6,709,671 disclose the invention of a water soluble film for oral administration with instant wettability. The film is comprised of at least one water-soluble polymer; at least one member selected from the group consisting of a polyalcohol, a surfactant and a plasticizer; at least one cosmetic or pharmaceutically active ingredient; and a flavoring agent. The existing coating technology is used to produce a film that exhibits instant wettability and rapid dissolution/disintegration upon administration in the oral cavity. In Example 5, the use of nicotine salicylate as an active substance is claimed. Physiologically acceptable films, including edible films and methods for producing them, are disclosed in U.S. Pat. Nos. 6,596,298, 6,923,981 and 7,025,983. The invention claims the use of the aforementioned films as a means to kill the plaque-producing germs that cause dental plaque, gingivitis and bad breath, using essential oils, such as thymol, methyl salicylate, eucalyptol and menthol.

Improved edible films for mucoadhesion produced with non-pullulan materials, such as maltodextrins, hydrocolloids and fillers are disclosed in U.S. Pat. No. 6,656,493; while a method of forming a thin film strip by coating a liner substrate with a wet slurry of film forming ingredients is claimed in U.S. Pat. No. 6,824,829.

Application WO 2003/026654 discloses a 3-layer composite film comprising nicotine dispersed in a water-soluble matrix, which may include a water-soluble gum and polyethylene glycol, sandwiched between two coating layers, for nicotine delivery to the oral cavity.

A film comprising a water-soluble polymer, polynucleotides and other purine and pyrimidine polymers, employed for masking the taste of a bitter medicament, and a bitter medicament, such as nicotine and other alkaloids, is disclosed in WO 2004/019885.

Water soluble films for delivering nicotine and treating nicotine addiction, comprising a pharmaceutically acceptable form of nicotine dispersed in a film consisting of a cellulose ether, a plasticizer and one or more additional ingredients, is described in WO 2006/114604. Here, the film is employed as a component of an enrobed tablet, a capsule, a multi-layered film, and a topical dosage form.

### SUMMARY OF THE INVENTION

Each of the film and coating production methods of the representative prior art patents, discussed briefly above, has certain disadvantages as compared to the production of the films and coatings of the present invention. These preceding methods often require specialized equipment, employ mixtures of several excipients, and may result in the degradation or evaporation of flavors. Still others are limited in the range of sizes that may be produced, as well as in the flavor loading as a percent of total material weight, as a result of the high temperatures sometimes employed.

It is an object of the present invention to overcome these disadvantages by providing a method to produce fast-dissolving films and coatings to use as a platform for flavors, food substances, active pharmaceutical ingredients, nicotine, as well as tobacco, consisting of a few simple, inexpensive, and easy-to-handle matrix materials. It is a further object of the present invention to provide a method of producing such materials using conditions sufficiently mild so as to minimize volatilization and degradation of the various components during processing. It is also another object of the present invention to provide a method to produce such materials that is more simple, more scalable and more economical in comparison to the prior art methods. These and other advantages of the present invention will become apparent to one skilled in the art with reference to the following descriptions.

Several test formulations of a water-soluble polysaccharide film containing methyl salicylate were tested for appearance, homogeneity, lack of self-adherence, flexibility and strength. Various polysaccharides, including alginates, carboxymethyl cellulose, pullulan and hydroxypropylmethyl cellulose (HPMC) were evaluated as primary film-forming agents. The effects of a series of emulsifying agents and plasticizers, including polysorbate 80, triethyl citrate, triacetin, propylene glycol and soy lecithin, on film characteristics were evaluated. These experiments led to the development of a model film formulation containing a minimum of excipients. This base formulation can be readily modified with preservatives, colors, emulsifiers and other excipients in order to optimize performance. The present invention may be practiced using a simple casting method, by moulding the polymeric solution on a simple Petri dish, or on a glass plate using a path wet film applicator or casting equipment (to obtain a more homogenous film thickness), or by more sophisticated extrusion machines, and by allowing it to dry at room temperature, or in an oven or in a ventilated oven, or in a vacuum chamber.

In contrast to previous methods, which require several excipients, including plasticizers and emulsifiers, the present invention may be practiced, and the desired release characteristics achieved, using a flavor and a single GRAS matrix material.

Moreover, even though a plasticizer is needed, the plasticizing properties of methyl salicylate may be advantageously used in the formulation. Film thickness may be controlled by simply using path wet film applicators of different path clearance adjustments, or by simple modification of polymer concentration and/or amount cast. Percent flavor loading is possible across a wide range simply by increasing or decreasing the amount of flavor incorporated into the casting matrix material. The flavor release rate may be controlled by previous encapsulation of the flavoring agent with GRAS materials. The same platform may also be used to deliver in the oral cavity an active pharmaceutical ingredient, such as nicotine for smoking cessation therapy; food substances, as well as tobacco. Nicotine, nicotine derivatives, food substances, as well as tobacco may be encapsulated before film preparation, as well. Moreover, the use of flavored films as wrapping matter for chewing tobacco is also claimed. Additional advantages of the present invention include the lack of organic solvents, inexpensive materials and unsophisticated equipment.

The present invention provides water-soluble film formulations, and methods of making and using the same. The edible films include at least one type of film forming agent other than pullulan that is readily available and at low cost. The simple water-soluble films described herein can be used to deliver materials to the oral cavity by themselves as stand alone materials, and they can be used to wrap, coat, bind and envelop other materials as part of an article for use in the oral cavity.

An advantage of the present invention is to provide edible films that include materials such as flavors, tobacco and nicotine, which may be used alone or as a component of another article for use in the oral cavity.

A further advantage of the present invention is to provide a method for delivery to the oral cavity that utilizes water-soluble film formulations which can effectively and rapidly release a material into the oral cavity upon contact with saliva.

Moreover, an advantage of the present invention is to provide a single, versatile film formulation that may then be used to produce a stand-alone film, and binders, coatings, and barriers as components of other articles for use in the oral cavity.

Additional features and advantages of the present invention are described herein, and will be apparent in the detailed description of the presently preferred embodiments and in the examples provided.

Applicants have uniquely discovered a simple and versatile film formulation that can be effectively utilized to prepare stand alone water-soluble films, films containing a variety of materials, and films that may be part of a composite article. The edible films are composed of ingredients that are readily available, can be prepared at lower costs, and display similar properties to those of the more complex, multi-component films composed of more expensive materials. In this regard, the water-soluble films described herein serve as physiologically acceptable films, that are readily adapted for use alone, and to adhere to or serve as components of composite articles and materials for delivery to the oral cavity, where they rapidly dissolve therein upon contact with saliva.

In this regard, these highly dissolvable films can act as a medium through which materials can be packaged and delivered to the oral cavity.

The edible films can include a variety of other suitable ingredients, such as plasticizers, colorants, flavoring agents, emulsifiers, surfactants, thickening agents, binding agents, sweeteners, fragrances, other like ingredients, and combinations thereof.

It is believed that the unique mixture of materials employed in the films of the present invention can provide a single, versatile base for producing both stand alone film compositions, as well as useful coatings, wrappers, binders and adjuncts to articles for use in the oral cavity.

As previously discussed, a variety of other suitable ingredients can be added to the films described in the present invention. These can include, for example, a pH control agent, such as urea and buffers; a saliva stimulating agent, including, for example, food acids such as citric, lactic, maleic, succinic, ascorbic, adipic, fumaric and tartaric acids; a biologically active agent, such as nicotine, surfactants, emulsifiers, plasticizers, preservatives, and colorants; and combinations thereof.

Any suitable amount and type of natural and/or synthetic food-grade emulsifiers can be used. For example, the emulsifier can include lecithin, food-grade non-ionic emulsifiers, fatty acids (C₁₀-C₁₈), mono and diacyl glycerides, ox bile extract, polyglycerol esters, polyethylene sorbitan esters, propylene glycol, sorbitan monopalmitate, sorbitan monostearate, sorbitan tristearate, other like emulsifiers, and combinations thereof.

The components of the film can be dispersed and mixed together using any appropriate mixing process, such as mechanical processing, vigorous stirring, turbulent flow, homogenization, sonication, colloid milling, and the like.

The present invention provides methods of producing the water-soluble film formulations. In general, the film formulations are prepared by first forming a base dispersion or solution that includes the primary film ingredients.

Along with the components of the base solution, additional ingredients, such as flavors, biologically active materials, emulsifiers, sweeteners, colorants, and combinations thereof, can be included.

In one embodiment, a film-forming dispersion is stirred continuously at room temperature, applied to an appropriate surface using any appropriate method, such as maceration, rolling, extrusion, spraying, dipping, deposing, and the like, and is then dried in any suitable manner, thereby forming the film. The entire process is conducted at a temperature below 40° C., thereby simplifying equipment requirements and minimizing volatilization and degradation of film components.

It should be appreciated that, given the simplicity and versatility of the film formulations described herein, any suitable type, number and order of process procedures and steps (i.e., mixing, heating, drying, cooling, addition of ingredients), process parameters (i.e., temperature, pressure, pH, process times) and the like, can be utilized.

### BRIEF DESCRIPTION OF THE DRAWINGS

FIG. 1 is a diagram of the process employed for fast-dissolving film preparation.

FIG. 2 is a representative image of the fast-dissolving films produced.

FIG. 3 is a graph illustrating methyl salicylate released in artificial saliva from the fast-dissolving films.

FIG. 4 is a bar graph illustrating the cumulative percent released methyl salicylate from fast-dissolving films in artificial saliva.

FIG. 5 is a diagram of an alternative process employed for fast-dissolving film preparation.

### DETAILED DESCRIPTION OF THE PREFERRED EMBODIMENTS

The invention will now be discussed by way of certain examples, which illustrate, but in no way limit, the invention.

### Example 1

### Fast-Dissolving Film Formulation

The model film formulation is composed of the following ingredients:

| | | | |
|---|---|---|---|
| Xylitol | 5%, w/w | (sugar) | 100 mg |
| Citric Acid | 5%, w/w | (siallorrheic/saliva-inducer) | 100 mg |
| Sodium Alginate | 5%, w/w | (stabilizer) | 100 mg |

Xylitol is a non-cariodenic “tooth-friendly” sugar. See, e.g., Saulo A. A. Food Safety and Technology, FST-16, January 2005; Hayes C. Journal of Dental Education 65(10).

These dry ingredients are levigated with 1 mL of 95% v/v ethanol and dissolved into: HPMC 60% (as 60 mL of a 2% w/v aqueous solution) 1200 mg

This aqueous phase is degassed (330 mg Hg) for 1 hour and added to an oil phase comprised of:

| | | |
|---|---|---|
| Propylene glycol | 5% | 100 mg |
| Methyl salicylate | 20% | 400 mg |

The mixture is homogenized with rapid agitation and deposed onto two 15 cm-diameter glass plates, then dried overnight at room temperature. The resulting film is smooth, pliable and does not self-adhere. Homogeneity studies were performed by dissolving randomly-sampled 1 cm2 squares, normalized by weight, in 50 mL aqueous methanol, then assaying the samples for methyl salicylate content by an HPLC method.

Methyl salicylate distribution in the model film is nearly homogeneous, varying by less than 1% between samples. Methyl salicylate content was approximately 1.0 mg/cm2, some 88% of the theoretical expected value. This simple formula has been readily modified with surfactants, plasticizers and other excipients, and can be customized for optimum performance.

For proof of concept, several examples of high dark snuff enveloped in the model film, and in strips of LISTERINE POCKETPACKS® for comparison, have been produced by sealing the wetted edges of the films around a 500 mg mass of snuff. Improved sealing techniques are required, as the water solubility of the films tends to produce weak, irregular seals. Heat annealing or a double sheet compression method would produce a suitable product.

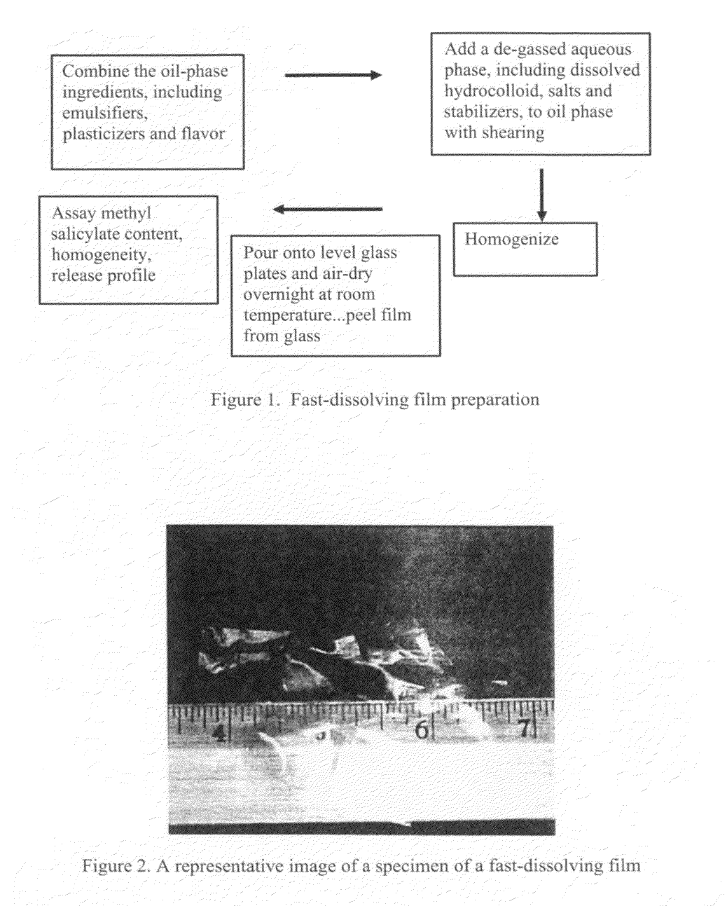

The process employed for film preparation is shown in FIG. 1, while a representative image of the films produced is illustrated in FIG. 2.

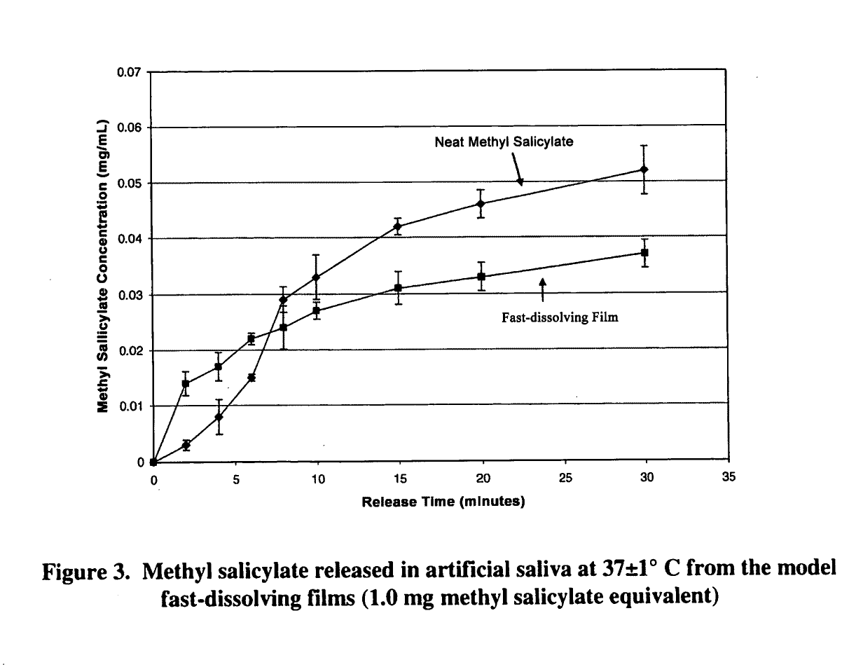

Methyl salicylate release from the model fast-dissolving film (20% w/w methyl salicylate) was assessed by placing a 1.0 cm apex square of homogeneous film (ca. 1 mg methyl salicylate equivalent) between layers of glass beads. Methyl salicylate release in artificial saliva at 37±1° C. was assessed and compared to release from neat controls, as shown in FIG. 3.

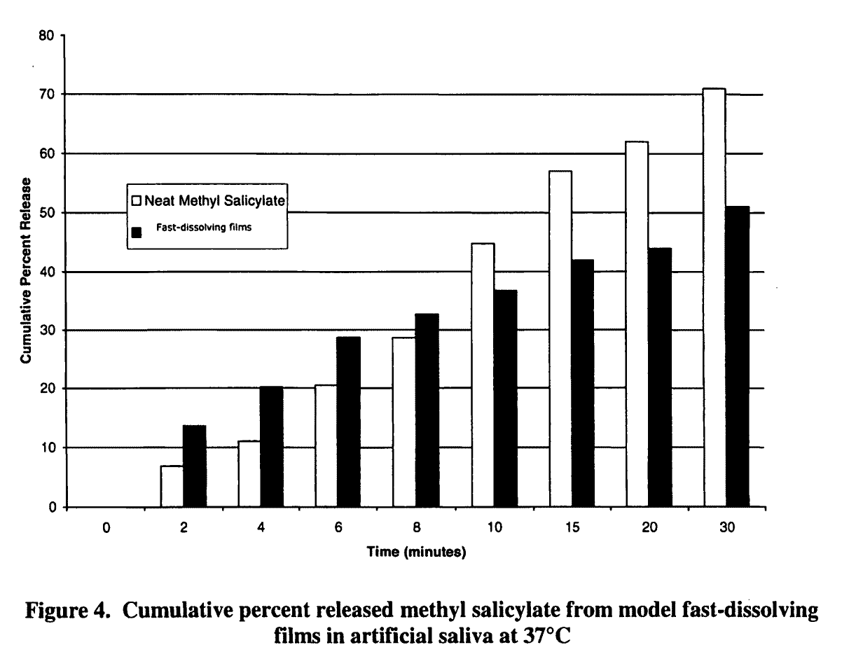

As compared to controls, methyl salicylate release from the fast-dissolving films showed an initial burst, followed by a slower release phase. Initial wetting at the film surface might facilitate an initial burst release, followed by slower diffusion through a hydrated polysaccharide hydrogel. Cumulative percent released methyl salicylate, as summarized in FIG. 4, reveals that approximately one quarter of encapsulated methyl salicylate is released within the first five minutes. These initial studies suggest that such polymer films, employed as fast-dissolving oral cavity platforms, coatings or films, offer an attractive alternative for sustained-release flavor formulation.

### Example 2

### Fast-Dissolving: Formulation Optimization

Two grades of water soluble cellulose ethers, e.g., hydroxyproylmethyl cellulose (HPMC), were selected as the main component of the film. Their characteristics and properties are reported in Table 1.

TABLE 1
characteristics of HPMC selected to prepare fast-dissolved films.

| | | | Viscosity of 2% |
|---|---|---|---|
| | Methoxyl content | Hydropropyl content | solution in |
| HPMC | (%) | (%) | water (mPa*s) |
| HPMC K4M | 19-24 | 7-12 | 2,308-3,755 |
| HPMC E4M | 28-30 | 7-12 | 3,000-5,600 |

Slight modifications on the previous formulations were carried out. The use of propylene glycol as a plasticizer was avoided because of its bad taste, and it was replaced with glycerin. In Tables 2 and 3, the composition of the formulations are reported, and the percentages are calculated on the basis of dry weight.

TABLE 2
The composition of the formulations based on HPMC E4M.

| | Content (%, w/w) | | | | | | |
|---|---|---|---|---|---|---|---|
| COMPONENTS | 1 | 3 | 4 | 6 | 7 | 8 | 11 |
| HPMC E4M | 35.22 | 65.22 | 51.53 | 42.85 | 23.12 | 22.73 | 24.61 |
| KOLLICOAT ® IR | — | 4.35 | 3.57 | 1.49 | 5.53 | 3.96 | 5.79 |
| glycerin | 4.35 | 4.35 | 3.57 | 1.49 | 2.01 | 0.99 | 2.13 |
| citric acid | 4.35 | 4.35 | 3.57 | 1.49 | 2.01 | 0.99 | — |
| xylitol | 4.35 | 4.35 | 3.57 | 1.49 | 2.01 | 1.98 | 2.13 |
| alginic acid | 4.35 | — | — | — | — | — | — |
| methyl salicylate | 17.39 | 17.39 | 34.18 | 51.18 | 65.33 | 69.35 | 65.33 |

TABLE 3
The composition of the formulations based on HPMC K4M.

| | Content (%, w/w) | | |
|---|---|---|---|
| COMPONENTS | 2 | 5 | 9 |
| HPMC K4M | 57.14 | 51.74 | 23.23 |
| KOLLICOAT ® IR | — | 3.48 | 5.05 |
| glycerin | 23.81 | 3.48 | 2.02 |
| citric acid | 7.14 | 3.48 | 2.02 |
| xylitol | 7.14 | 3.48 | 2.02 |
| methyl salicylate | 4.76 | 34.33 | 65.66 |

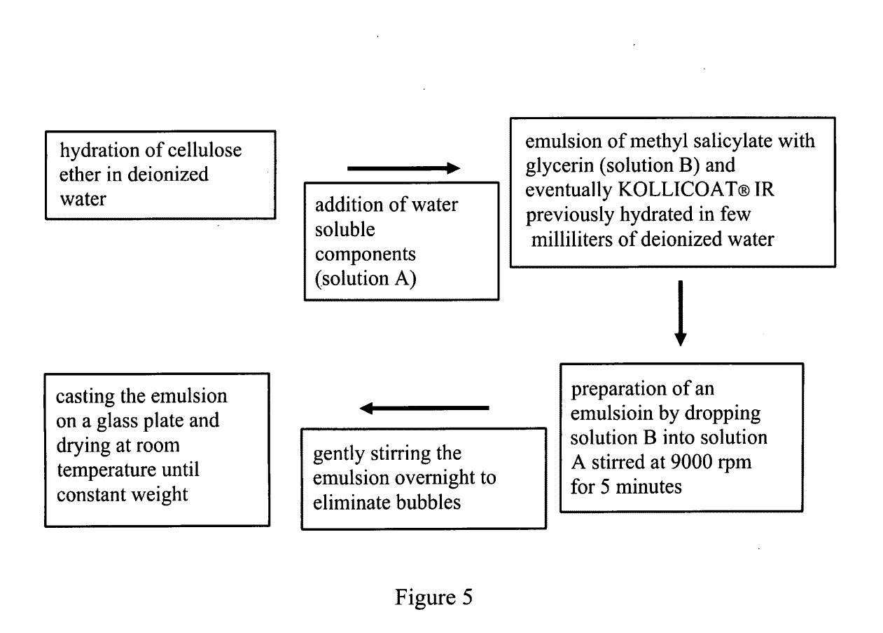

The preparation of film included the steps reported in FIG. 5.

A few days after preparation, the films underwent a visual inspection to evaluate flexibility and evidence of any defects, such as bubbles, pores, lack of uniform thickness and oily appearance. The methyl salicylate content was evaluated only for the films that were visually homogeneous. There was no evidence of any defects.

Component formulations #1 and #2 were discarded because they assumed an oily texture which could be due to the diffusion of methyl salicylate after few days of storage at room temperature.

To increase the dissolution rate of the polymeric matrix, low-swellable ingredients, such as alginic acid, were replaced by KOLLICOAT® IR, a polyvinyl alcohol-polyethylene glycol graft copolymer, used as an instant release coating in the pharmaceutical field. Formulation #3 appeared to be more stable over time because of the presence of KOLLICOAT® IR, which can act as a stabilizer for the emulsion. To confirm this feature, the content of methyl salicylate was increased and two different types of HPMC, namely HPMC E4M (formulations #4 and #6) and HPMC K4M (formulation #5), were chosen to prepare the films. The MS content in Formulation #4 and 6 was 9.6 μg/cm2 and 118.4 μg/cm2, respectively. Formulation #5 was discarded because after drying, wrinkles and white blots were evident.

The influence of drying temperature on the methyl salicylate content was also evaluated. Formulation #4 was drier at high temperature, namely 40° C. and 55° C., and samples were analyzed for methyl salicylate content. A drying temperature higher than 40° C. greatly affected the MS content (9.6 μg/cm2 at room temperature vs. 2.7 μg/cm2 at 55° C.). Nevertheless, the high variability in methyl salicylate content of the film dried at high temperature could be due to the lack of homogeneity in thickness of the films.

In formulation #7, the amounts of HPMC E4M and KOLLICOAT® IR were modified, while keeping constant the amount of the plasticizers. A further attempt to increase the methyl salicylate content was carried out. The film was uniformly transparent and presented good flexibility. During the storage at room temperature, these properties were maintained even if there was a slight tendency to roll up due to the thinness. The average content of methyl salicylate was about 1750 μg/cm2. Nevertheless, the method of casting, i.e., pouring the mixture on a glass Petri dish and drying it on an uneven surface, did not provide uniform content of methyl salicylate. This problem might be overcome by laminating the mixture on a suitable backing layer using a film casting knife.

After increasing the content of methyl salicylate in formulation #8, a separation of phase in the emulsion occurred. In formulation #9, HPMC E4M was replaced by HPMC K4M, but even when the methyl salicylate content was higher (3480 μg/cm2) than that in formulation #7, the use of HPMC E4M was preferred because the resulting film appeared more flexible and elegant.

To reduce the tendency to roll up, one of the plasticizers, namely citric acid, was removed (formulation #11) and the ratios among the other constituents were kept constant. The film maintained good flexibility without folding over time. The experimental loading of methyl salicylate was 11%.

The stability of methyl salicylate in Formulation #11 was evaluated in terms of amount of methyl salicylate entrapped within the film over time as well as the chemical stability. After one week of storage at room temperature, methyl salicylate content was lowered 10-fold, and the presence of the degradation product, namely salicylic acid, was evident. When samples were sealed in plastic bags and stored at 4° C., the methyl salicylate loaded in the film was 50% of the original value.

Formulation # 11 was further modified in order to incorporate other active pharmaceutical ingredients such as nicotine free base, nicotine tartrate, or other nicotine salts and derivatives, tobacco and mixtures thereof. Furthermore, methods to encapsulate the aforementioned actives, in order to modify their release, are described as well.

First of all, fast-dissolving films were prepared using the same composition of previously developed formulation 11 (without methyl salicylate) using a new casting method. An 8-Path Wet Film Applicator and a glass plate were employed to cast the films in order to obtain homogenous film thickness. Different actual path depths, ranging from 1 to 50 Mils, were assessed. In particular, the film thickness after drying and the peel-off properties were evaluated to select the most suitable path depth.

The 50 Mils (˜1.25 mm) path was found to be the most appropriate because of the final film thickness and flexibility, even though a slight increase in HPMC concentration (from 2.5 to 3% w/v) was needed in order to produce higher viscosity during the preparation procedure. In all cases, the peel-off was somewhat difficult. To improve the film detachment from the glass plate, a thin layer of paraffin oil was deposited on the glass. Film composition is reported in the following table (Table 4).

TABLE 4
Composition of the optimize film.

| | | Film at 3% of HPMC | |
|---|---|---|---|
| | Film at 3% of HPMC | (composition for 100 | |
| | (dry weight) | mL) | |
| HPMC (E4M) | 71.1% | 3 | g |
| KOLLICOAT IR | 16.7% | 0.7058 | g |
| Glycerin | 6.1% | 0.2596 | g |
| Xylitol | 6.1% | 0.2596 | g |
| Distilled water | — | 100 | g |

The films were prepared as follows. Briefly, 3% (w/v) of HPMC (E4M) was swelled in distilled water (at least 24 hours under magnetic stirring). Then, all the other components were added and mixed until dissolution. The gel was left at room temperature until all the embedded air bubbles were gone. The gel was slowly dropped onto the glass plate and the wet film applicator was driven over. The film was cast under the fume hood over night.

A piece of blank film (3% of HPMC) of 48 cm² weighed 0.1894 g (corresponding to ˜4.5 mL of wet gel). A film size of 6 cm² (3×2 cm) was considered desirable (blank film weight ˜25 mg).

The optimized film formulation was loaded with nicotine in different forms, namely nicotine free base, nicotine hydrogen tartrate, and snuff. In an analogous fashion to commercial nicotine chewing gums, 1 mg of nicotine per piece (corresponding to 2.85 mg of nicotine hydrogen tartrate or 100 mg of snuff) was used as a target loading. The films loaded with nicotine free base and hydrogen tartrate were as flexible as the blank ones and dissolved within 2 minutes in artificial saliva at 37° C.

Snuff was embedded into the film as whole snuff and as sieved snuff (with a particle size lower than 106 μm). The film containing the whole snuff was not suitable since it was less flexible than the blank one and not homogeneous. On the contrary, sieved snuff allowed a homogeneous dispersion of snuff particles that made the film flexible and then suitable for further investigations.

### Example 3

### Nicotine-Cetyl Alcohol Microparticles Preparation

Nicotine free base has been embedded within the cetyl alcohol microparticles developed as delivery system for food flavors. Cetyl alcohol (1.6 g) and nicotine free base (0.4 g) were heated at 65° C. in a 20 mL scintillation vial under magnetic stirring. The melted internal phase was injected (via a plastic syringe) into 800 mL of deionized water (65° C.) containing 0.35% poly (vinyl alcohol) and agitated with mechanical stirring (500 rpm). The vessel was equipped with an appropriate baffle system. Five minutes after injection the heating was discontinued and the dispersion was cooled by recirculation of ice cold water. Particles were recovered by filtration, washed with 3 L of deionized water, and dried under vacuum overnight (15 mm Hg). The production method had a yield of 70%.

### Example 4

### Snuff Cetyl Alcohol Microparticles Preparation

Snuff particles have been embedded within the cetyl alcohol microparticles, as well. The above-mentioned encapsulation procedure, slightly modified, was employed to encapsulate snuff particles with a particle size lower than 106 μm (obtained by sieving the whole snuff). Due to the low snuff nicotine content, a larger target loading and batch size were used. Briefly, 5 g of cetyl alcohol and 2.5 g of snuff were mixed in a small beaker and heated at 65° C. to melt the cetyl alcohol. The mixture was magnetically stirred for 2 minutes to produce a homogenous snuff dispersion. The melted dispersion was injected via a plastic syringe into 1 L of deionized water (65° C.) containing 0.35% poly (vinyl alcohol) stirred at 500 rpm. The rest of the procedure was the same as reported in Example 3. During particle filtration it was noticed that the filtered external phase was brown in color. Probably, the hot cetyl alcohol was able to extract (during melting and stirring before injection) some of the natural or artificial snuff colors that subsequently diffused into the external phase. The encapsulation method yield was around 65%. The low microparticles recovery was in part due to some internal phase loss during the operation of withdrawing and injection. In fact, the melt had a relatively high amount of dispersed solid material (about 33%) that made it more viscous as compared to cetyl alcohol/nicotine or cetyl alcohol/methyl salicylate.

### Example 5

### Nicotine Poly(Lactide-Co-Glycolide) Microparticles Preparation

Nicotine and snuff particles have been encapsulated in RESOMER® RG 502H poly(lactide-co-glycolide) (PLGA) microparticles.

Briefly, 200 mg of nicotine free base and 800 mg of PLGA were dissolved in 8 g of methylene chloride and injected via a glass syringe (by way of a needle) into 800 mL of deionized water containing 0.35% of poly (vinyl alcohol). The injection was performed at 4° C. under mechanical agitation (1500 rpm). One minute after injection the stirring was lowered at 1000 rpm, while the temperature was kept at 4° C. for 5 minutes. Then, the temperature was increased to 25° C. (within 10 minutes), left at 25° C. for 5 minutes, and then increased to 40 (over 10 minutes). The temperature was maintained at 40° C. for 40 minutes and then lowered to room temperature. Particles were recovered by filtration, washed with 3 L of deionized water, and freeze dried over night. The procedure had a yield of 75.6%. Particles were freely flowing immediately after lyophilization, while the following day they were clumped together in a cake. This can be reasonably ascribed to a lowering of the glass transition temperature due to plasticization and/or polymer degradation. In fact, a reduction in the average molecular weight (due to polymer degradation during microparticles production) can be hypothesized because of the presence of a tertiary amine in the nicotine structure. The basic pyridinyl moiety may also contribute to polymer degradation

### Example 6

### Snuff Poly(Lactide-Co-Glycolide) Microparticles Preparation

A similar preparation method was employed for snuff encapsulation. One gram of PLGA, 0.5 g of snuff (particle size lower than 106 μm) and 10 g of methylene chloride, comprised the internal phase, while 1 L of 0.35% of poly (vinyl alcohol) water solution constituted the external phase. Other parameters were unchanged. This preparation method gave a yield of 78.3%. Snuff PLGA microparticles were not sticky like nicotine microparticles.

It should be understood that various changes and modifications of the presently preferred embodiments described herein will be apparent to those skilled in the art. Such changes and modifications can be made without departing from the spirit and scope of the present invention and without diminishing its intended advantages. It is therefore intended that all such changes and modifications be covered by the appended claims.

We claim:

1. A method for producing fast-dissolving films for use as platforms for the delivery of material to the oral cavity, comprising the steps of
combining an oil-phase component of emulsifiers and plasticizers, including methyl salicylate, with a de-gassed aqueous-phase component, including dissolved hydrocolloid, salts and stabilizers;
homogenizing the resulting mixture with rapid agitation;
deposing the homogenized mixture upon a substrate; and
drying the homogenized mixture at a temperature below 40° C.

2. A method for producing fast-dissolving films for use as platforms for the delivery of material to the oral cavity, comprising the steps of
hydrating water soluble cellulose ethers in water;
emulsifying methyl salicylate with glycerin;
dropping the emulsion of methyl calicylate and glycerin into the hydrated ethers with rapid stirring;
de-gassing the resulting emulsion;
deposing the de-gassed emulsion onto a substrate; and
drying the de-gassed emulsion at a temperature below 40° C.

3. The method of claim 1 or 2, wherein said material is selected from the group consisting of a flavor, a flavor dispersed in a particulate matrix, a material derived from tobacco, a material derived from tobacco dispersed in a particulate matrix, a biologically active material, a biologically active material dispersed in a particulate matrix, and mixtures thereof.

4. The method of claim 3, wherein said flavor comprises from about 5% to about 80% dry weight of the fast-dissolving film, and is selected from the group consisting of a volatile oil, an essential oil, a botanical extract, methyl salicylate, ethyl salicylate, cinnamic acid, cinnamon oil, peppermint oil, spearmint oil, wintergreen oil, acetaldehyde, acetoin, aconitic acid, anethole, benzaldehyde, N-butyric acid, d- or 1-carvone, cinnamaldehyde, citral, decanal, diacetyl, ethyl acetate, ethyl butyrate, ethyl vanillin, eugenol, geraniol, geranyl acetate, glycerol tributyrate, limonene, linalool, linalyl acetate, 1-malic acid, methyl anthranilate, 3-methyl-3-phenyl glycidic acid ethyl ester, piperonal, vanillin, citrus flavoring, berry flavoring, and mixtures thereof.

5. The method of claim 3, wherein said material derived from tobacco is selected from the group consisting of raw tobacco, cured tobacco, snuff, moist snuff, snuss, an extract of tobacco, and mixtures thereof.

6. The method of claim 3, wherein said particulate matrix is selected from the group consisting of a wax, paraffin, a fatty alcohol, a fatty acid, a monoglyceride, a diglyceride, a triglyceride, lecithin, cholesterol, a polylactide, a polyglycolicide, PLGA, an edible polymer, and mixtures thereof.

7. The method of claim 3, wherein said biologically active material is selected from the group consisting of a pharmacologically active form of nicotine, a pharmaceutically acceptable salt of nicotine, and mixtures thereof.

8. A fast-dissolving film for use as a platform for the delivery of material to the oral cavity, comprising a film forming agent; a plasticizing agent; and a fast-dissolving, water-soluble agent.

9. The fast-dissolving film of claim 8, wherein said film forming agent comprises from about 2% to about 50% dry weight of the film, and is selected from the group consisting of a cellulose, a cellulose ether, a starch, a modified starch, a natural gum, an edible polymer, a plant extract, an algae extract, a hydrocolloid flour, and mixtures thereof.

10. The fast-dissolving film of claim 8, wherein said plasticizing agent comprises from about 0.2% to about 20% dry weight of the film, and is selected from the group consisting of methyl salicylate, glycerin, propylene glycol, a polysorbate, a polyethylene glycol, a polyol, lecithin, a sugar syrup, a starch hydrolysate, a triacyl citrate, triacetin, and mixtures thereof.

11. The fast-dissolving film of claim 8, wherein said fast-dissolving, water-soluble agent comprises from about 1% to about 30% dry weight of the film, and is selected from the group consisting of a polyethylene glycol, a polyol, a polyol-polyethylene glycol graft co-polymer, and mixtures thereof.

12. The fast-dissolving film of claim 8, and further comprising an additional agent comprising from about 0.1% to about 30% dry weight of the film that is selected from the group consisting of a sweetener, a saliva-generating agent, a colorant, an emulsifier, a preservative, and mixtures thereof.

13. The fast-dissolving film of claim 12, wherein said sweetener is selected from the group consisting of xylitol, mannitol, sorbitol, a sugar, a sugar alcohol, a sugar syrup, molasses, aspartame, acesulfame, cyclamate, sucralose, and mixtures thereof.

14. The fast-dissolving film of claim 12, wherein said saliva-generating agent is selected from the group consisting of citric acid, tartaric acid, ascorbic acid, fumaric acid, malic acid, maleic acid, succinic acid, and mixtures thereof.

15. The fast-dissolving film of claim 8, wherein said material is selected from the group consisting of a flavor, a flavor dispersed in a particulate matrix, a material derived from tobacco, a material derived from tobacco dispersed in a particulate matrix, a biologically active material, a biologically active material dispersed in a particulate matrix, and mixtures thereof.

16. The fast-dissolving film of claim 15, wherein said flavor comprises from about 5% to about 80% dry weight of the fast-dissolving film, and is selected from the group consisting of a volatile oil, an essential oil, a botanical extract, methyl salicylate, ethyl salicylate, cinnamic acid, cinnamon oil, peppermint oil, spearmint oil, wintergreen oil, acetaldehyde, acetoin, aconitic acid, anethole, benzaldehyde, N-butyric acid, d- or 1-carvone, cinnamaldehyde, citral, decanal, diacetyl, ethyl acetate, ethyl butyrate, ethyl vanillin, eugenol, geraniol, geranyl acetate, glycerol tributyrate, limonene, linalool, linalyl acetate, 1-malic acid, methyl anthranilate, 3-methyl-3-phenyl glycidic acid ethyl ester, piperonal, vanillin, citrus flavoring, berry flavoring, and mixtures thereof.

17. The fast-dissolving film of claim 15, wherein said material derived from tobacco is selected from the group consisting of raw tobacco, cured tobacco, snuff, moist snuff, snuss, an extract of tobacco, and mixtures thereof.

18. The fast-dissolving film of claim 15, said particulate matrix is selected from the group consisting of a wax, paraffin, a fatty alcohol, a fatty acid, a monoglyceride, a diglyceride, a triglyceride, lecithin, cholesterol, a polylactide, a polyglycolicide, PLGA, an edible polymer, and mixtures thereof.

19. The fast-dissolving film of claim 15, said biologically active material is selected from the group consisting of a pharmacologically active form of nicotine, a pharmaceutically acceptable salt of nicotine, and mixtures thereof.

20. A method for producing fast-dissolving films for use as platforms for the delivery of material to the oral cavity, comprising the steps of
dispersing a film forming agent, a plasticizer and a fast-dissolving soluble agent in water by stirring, milling, swelling, or homogenizing to accomplish a thorough mixing, thereby forming a film-forming composition as a freely flowing liquid at a temperature below 40° C.;
applying the film forming composition to a substrate by deposing, casting, macerating, rolling, spraying, extruding, or combinations thereof; and
allowing the film-forming composition to dry at a temperature below 40° C.
